(19) United States
(12) Patent Application Publication
(10) Pub. No.: US 2014/0214008 A1
(43) Pub. Date: Jul. 31, 2014

(54) Precision Venting Arrangement for Pressure Equilibration to Enhance Drainage of Medical Tubing

(71) Applicants: Steven J. Babb, Seminole, FL (US); David W. Underwood, Tampa, FL (US); Dennis M. Kay, Largo, FL (US)

(72) Inventors: Steven J. Babb, Seminole, FL (US); David W. Underwood, Tampa, FL (US); Dennis M. Kay, Largo, FL (US)

(21) Appl. No.: 13/751,397

(22) Filed: Jan. 28, 2013

(51) Int. Cl. A61M 25/00; A61F 5/441

(52) U.S. Cl. CPC A61M 25/0017; A61F 5/441
USPC 604/544; 604/347

(57) ABSTRACT

A precision venting arrangement for urinary drainage includes a tubular housing communicating with medical tubing receiving urine from the body and a pouch for storing the urine, the tubular housing having opposing open ends with a lumen passing longitudinally therebetween, an intermediate portion disposed between the open ends and an intermediate portion with a side passage disposed at the intermediate portion and extending through a side wall of the housing with a venting valve disposed in the side passage having a biased dosed distal end and an open proximal end exposed to atmospheric pressure such that the venting valve is opened when the differential between the pressure in the lumen of the housing and atmospheric pressure exceeds a predetermined value.

### CROSS-REFERENCE TO RELATED PATENT APPLICATION

The subject patent application is a continuation of U.S. patent application Ser. No. 12/987,867 filed Jan. 1, 2011, the entire disclosure of which is incorporated herein by reference.

### BACKGROUND OF THE INVENTION

1. Field of the Invention

The present invention relates to medical tubing drainage and, more particularly, to a precision venting arrangement for reducing the potential for accumulation of negative pressure within the lumen of medical tubing.

2. Brief Discussion of the Related Art

Medical procedures, systems and devices frequently require gravity draining of bodily fluids. Examples of such procedures, systems and devices include the draining of urine through indwelling catheters, particularly “Foley” catheters, drainage of urine through external continence devices and other gravity drainage arrangements. Hydrostatic forces produced by urine drainage are capable of causing discomfort, irritation and/or inflammation of tissue, particularly the perimeatal area (tip) of the penis. This phenomenon is referred to as a “siphon effect” when urine collection assemblies are discussed, for example in U.S. patent application Ser. No. 10/961,396, the disclosure of which is incorporated herein by reference. As described in the '396 application, the siphon effect is a barometric phenomenon whereby negative pressure (partial vacuum) accumulates in urinary drainage tubing and collectors. It has been found that excessive negative pressure (partial vacuum) within the lumen of a drainage catheter can cause tissue damage. For example, excessive negative pressure in an external urinary catheter can injure perimeatal tissue while excessive negative pressure in an internal catheter can cause tissue damage to the lining of the bladder. Accordingly, it would be desirable to reduce the negative pressure to a safe level by venting the system (tubing) while dosing the system when the negative pressure is relieved to provide a leak-tight seal.

### SUMMARY OF THE INVENTION

Accordingly, it is a primary aspect of the present invention to prevent excessive negative pressure in tubing of a medical drainage system by utilizing a precision venting arrangement disposed between a catheter draining fluid from the body and a pouch for collecting the fluid.

In another aspect, the present invention involves a precision venting arrangement for use with medical tubing including a duckbill venting valve disposed in a tubular housing communicating with a source of bodily fluid and a pouch for collecting the bodily fluid where the duckbill venting valve opens at a predetermined differential (negative pressure) between the pressure in the tubing arrangement and atmospheric pressure.

The precision venting arrangement of the present invention employs a vent device that can be installed inline in any medical drainage catheter and does not require construction of a separate container or bag to mount the vent. The precision venting arrangement employs a vent valve that positively doses upon completion of the venting cycle due to preloading, i.e. biasing of the valve sealing surfaces and is, thus, resistant to leakage from capillary action. No filter media is required to be assembled over the vent valve such that leakage associated with filter media from capillary action is not present and no plugging of filter media results from salt residue, for example from evaporating urine. The duckbill vent valve of the precision venting arrangement provides precise control of partial vacuum without requiring a carefully matched, costly and less desirable molded sealing surface, and the lumen of the housing of the precision venting arrangement can accommodate passage of an intermittent catheter. The precision venting arrangement reduces the risk of tissue damage or infection of damaged tissue including capillary rupture, cell disruption, hemorrhage and/or ischemia.

The precision venting arrangement of the present invention includes a venting valve, a housing and tubing connections having various configurations according to mating tubing/catheters. The venting valve design provides precision opening and dosing of the vent and permits repeated and reliable opening and dosing in a narrow range of negative pressure to provide a seal with minimized leakage.

A particularly advantageous implementation of the precision venting arrangement of the present invention utilizes a vent device including a duckbill valve for venting to provide a predetermined, controlled opening and dosing of the vent. The characteristics of the duckbill valve are particularly suited to the desired requirements of gravity drainage tubing arrangements and, in particular, has an opening vacuum less than four inches H₂O to substantially eliminate the siphon effect while also preventing leakage or capillary action through the sealing surfaces of the valve. The duckbill valve has opposing, elastomeric sealing surfaces which are precisely cut apart, typically with a razor, to precisely and conformably mating configurations such that the duckbill valve does not require a secondary sealing surface.

The housing of the precision venting arrangement of the present invention is tubular, essentially in the form of a “tee” with opposing open ends configured to accommodate elastomeric tubing via a friction fit and a side port with a countersunk hole or passage to accommodate a duckbill valve with tight tolerances for precision opening and closing pressures. A retaining cap fits over the side port and can have a cross pattern such that the venting valve would not be mistaken by a practitioner as a sample or injection port and such that the likelihood of occlusion by a patient's adipose tissues is prevented.

When a closed drainage system is utilized, such as for Foley catheter drainage, the precision venting arrangement of the present invention can be fitted with an antimicrobial filter which would remain dry at all times with the duckbill valve performing all of the venting functions and keeping the filter (membrane) free of capillary action and prevent potential plugging from dried urine salts.

Other aspects and advantages of the present invention will become apparent from the following description of an embodiment of the present invention taken in conjunction with the accompanying drawings.

### BRIEF DESCRIPTION OF THE DRAWINGS

FIG. 1 is an elevation of a precision venting arrangement according to the present invention.

FIG. 2 is an exploded view, in perspective, of the precision venting device according to the present invention.

FIG. 3 is an exploded end view of the precision venting device according to the present invention.

It has been found that excessive negative pressure (partial vacuum) within the lumen of a drainage catheter can cause tissue damage. For example, excessive negative pressure in an external urinary catheter can injure perimeatal tissue while excessive negative pressure in an internal catheter can cause tissue damage to the lining of the bladder.

FIG. 4 is an exploded side view of the precision venting device according to the present invention.

FIGS. 5, 6 and 7 are perspective, side and end views of a duckbill vent valve for use with the precision venting device of the present invention.

FIG. 8 is a cross-sectional of the precision venting device of the present invention with the vent valve positioned in a side passage of the precision venting device housing.

### DETAILED DESCRIPTION OF THE INVENTION

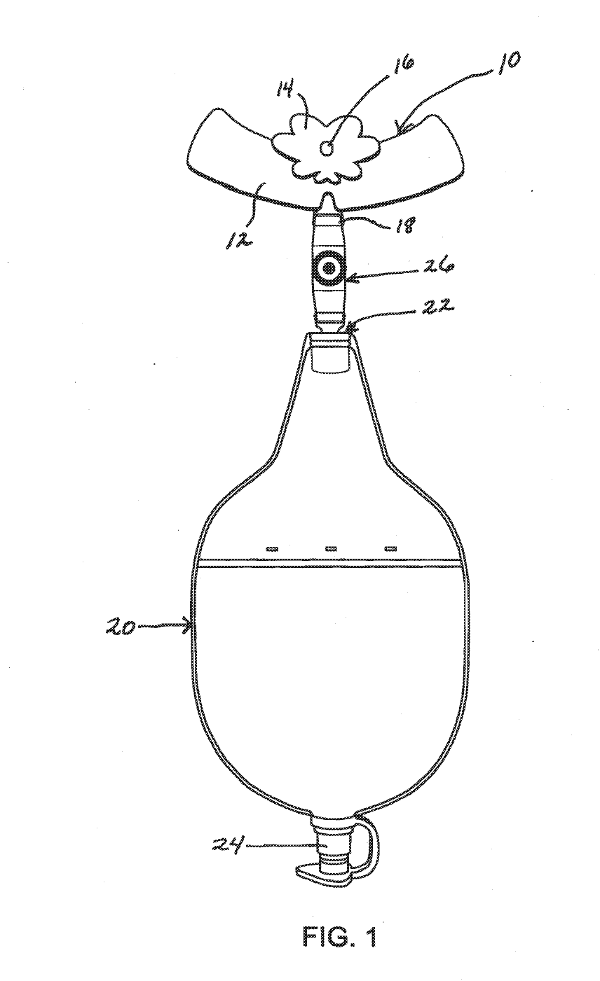

A precision venting arrangement according to the present invention is illustrated in FIG. 1 and includes a catheter device 10 for attachment to a penis for collecting urine from the meatus or urinary opening at the end of a penis as is well known and referred to as a male external catheter such as that marketed by BioDerm, Inc. of Largo, Fla. The catheter device includes an adhesive seal arrangement 12 for securement to the head of a penis and a faceplate 14 for engaging the tip of a penis. The faceplate 14 has an opening 16 therein for alignment with the urinary opening or meatus. In use, the external catheter device 10 is secured to the penis and urine passes through a tubular stem 18 extending from a housing of the external catheter device. The precision venting arrangement of the present invention can be used with any medical tubing for gravity drainage, and the external catheter device 10 illustrated is for exemplary purposes only. To this end, it should be appreciated that the precision venting arrangement of the present invention can be used with other medical tubing or catheters, for example Foley catheters, which are well known and inserted within the urethra.

A pouch 20 has an upper or inlet port 22, a bladder for collecting urine and an outlet port 24 for removing urine from the pouch. The pouch 20 can have any desirable configuration, and reference is made to the previously referred to patent application Ser. No. 10/961,396 for more detailed discussion of a specific pouch for use with the present invention.

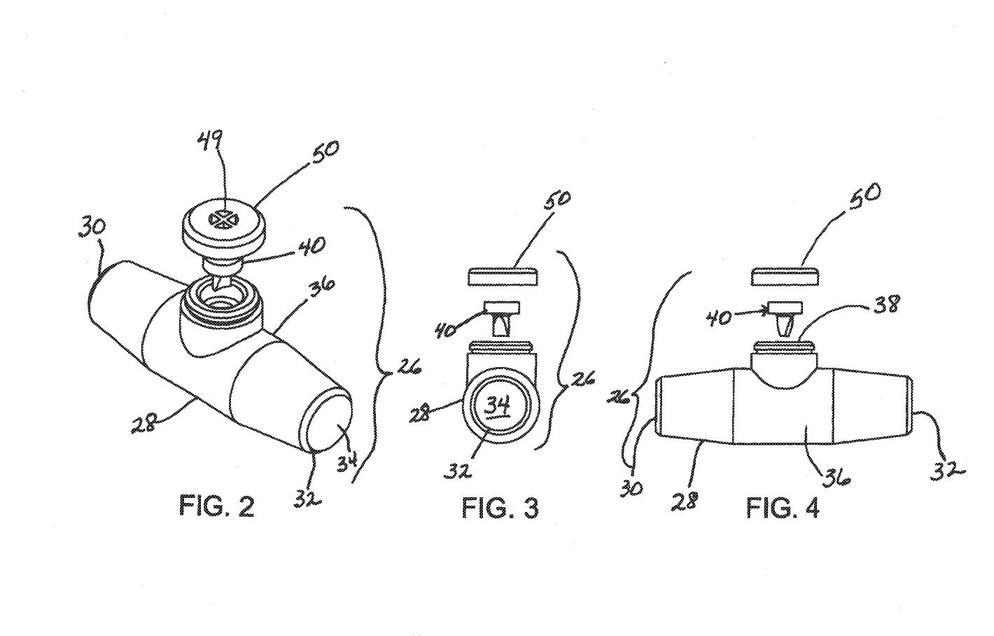

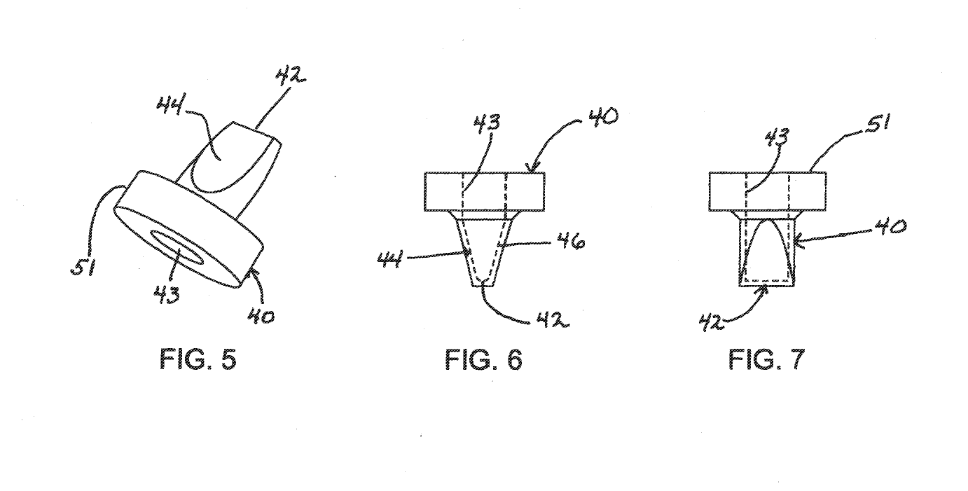

A precision venting device 26 is shown in FIGS. 1-4 and communicates with the tubular stem 18 of the external catheter 10 and the inlet port 22 of the pouch 20. The precision venting device 26 is formed of a tubular housing or body 28 having first and second opposing open ends 30 and 32, respectively, communicating with a lumen 34 passing longitudinally through the housing. Looking at FIG. 1, open end 30 forms an inlet opening for the vent device and opening 32 forms an outlet opening for the vent device such that urine passes from the medical tubing (external catheter device 10) through the lumen 34 of the housing 28 to exit into pouch 20. The housing 28 has an intermediate portion 36 disposed between the open ends 30 and 34, and the housing has a “T” shape to form a side passage 38 extending through a side wall of the housing to communicate with the lumen 34 at an angle thereto, the angle shown being 90°. A venting valve 40 is disposed in the side passage 38 such that a distal end 42 of the venting valve is exposed to pressure in the lumen 34, and a proximal end 43 of the venting valve is exposed to atmospheric pressure.

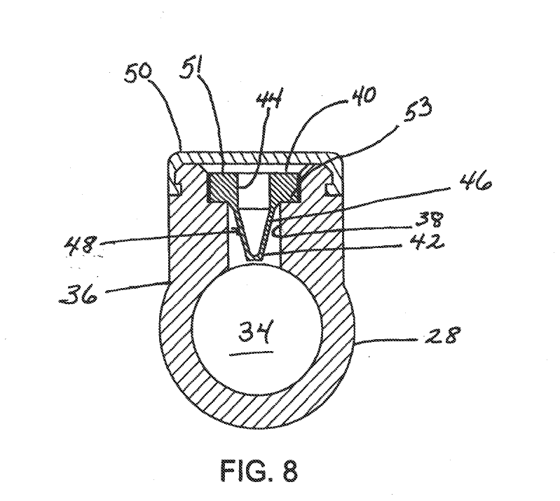

The venting valve 40 has a duckbill configuration with flaps 44 and 46 biased toward each other so that the distal end 42 is normally dosed or sealed. As shown in FIG. 8, the open proximal end 43 of the duckbill valve is exposed to atmospheric pressure via apertures 49 in a cap 50 snapped onto the intermediate portion 36, the cap 50 holding the duckbill valve in position. As best shown in FIG. 8, the duckbill valve is disposed entirely in the intermediate portion of the housing with a collar 51 received on a countersunk shoulder 53 adjacent side passage 38 in a transverse portion of the housing so as not to extend into the lumen 34 of the housing and to not disrupt flow of urine therethrough. The recessed positioning of the distal end 42 of the duckbill valve permits optional intermittent catheterization through the precision vent device in that the flow path for urine is dear of obstructions. The vent valve 40 will open at a predetermined pressure differential between the pressure in the lumen 34 and atmospheric pressure such that residual negative pressure in the medical tubing (catheter) can be minimized, and from a practical standpoint to less than 4 inches H₂O (10 cm H₂O) relative to atmospheric pressure. In operation, the vent valve will open and admit air through the apertures 49 in cap 50 to break any partial vacuum whenever the vacuum exceeds the predetermined negative pressure, such as upon passage of urine through the tubing. The vent valve will remain open until sufficient air is introduced into the tubing to relieve the partial vacuum again (i.e. to less than 4 inches H₂O). When the partial vacuum has been relieved, the vent valve will dose until the next cycle is initiated by fluid flow, gravity urine flow leading to excessive negative pressure.

The duckbill vent valve is made of an elastomeric material such that the vent valve is preloaded, or biased, and is resistant to leakage. The duckbill valve has opposing, elastomeric sealing flaps or surfaces which, during manufacture, are precisely cut apart, typically with a razor, and thereby precisely and conformably mate such that the duckbill vent valve does not require a secondary sealing surface in the device.

The housing 28, accordingly, forms a tubing adapter in the configuration of a “T” which is able to accommodate elastomeric tubing from the catheter device and from the pouch by friction fit. The side passage or port has a countersunk hole to accommodate the duckbill vent valve with tight tolerances for precision opening and dosing pressures. The retaining cap has a cross pattern on the outer surface thereof adjacent the apertures, with the cross pattern forming the apertures and also preventing the device from being mistaken as a sample or injection port and further reducing the likelihood of occlusion by adipose tissues.

A dear, shrink wrap can be applied to the precision valve device by affixing the shrink wrap to the tubing extending from the catheter device and the pouch.

An example of materials that can be utilized to form the precision vent device are, for the tubular housing (body) and retainer cap, high density polyethylene (HDPE) or acrylonitrile butadiene styrene (ABS) or other semi-rigid or rigid plastics suitable for use in medical devices that have modest elasticity to permit the inner snap ring of the cap to snap over the intermediate portion of the body without stress damage and will provide sufficient friction to grip the tubing from the catheter device and the pouch when attached. Tests of the above materials with common urinary drainage tubes have exceeded 20 pounds yield strength. The duckbill vent valve is made of an elastic material such as synthetic rubber, ethylene propylene diene monomer (EPDM) or silicone rubber or alternative elastomers that, in [a] combination with the specific dimensions and geometry of the vent valve provide elasticity sufficiently high to open and close the flaps of the duckbill vent valve at a very low and controllable vacuum range with low durometer sufficient to optimize the sealing of the two opposing duckbill surfaces or flaps.

Inasmuch as the present invention is subject to many variations, modifications and changes in detail, it is intended that all subject matter discussed above or shown in the accompanying drawings be interpreted as illustrative only and not be taken in a limiting sense.

What is claimed is:

1. A precision venting arrangement for urinary drainage comprising
medical tubing for receiving urine from the human body;
a tubular housing having first and second opposing open ends communicating with a lumen passing longitudinally through said housing, an intermediate portion disposed between said first and second open ends, and a side passage disposed at said intermediate portion extending through a side wall of said housing to communicate with said lumen at an angle thereto, said first open end communicating with said medical tubing; and
a venting valve disposed in said side passage of said housing, said venting valve having a biased closed distal end and an open proximal end exposed to atmospheric pressure, said distal end of said venting valve being opened when the differential between the pressure in said housing lumen and atmospheric pressure exceeds a predetermined value.

2. A precision venting arrangement as recited in claim 1 wherein said venting valve includes a duckbill valve having opposing flaps biased toward each other to form a seal at said distal end during normal operation and to open when the pressure in said housing lumen drops below a predetermined value and to close when the pressure in said housing lumen exceeds a predetermined value.

3. A precision venting arrangement as recited in claim 2 wherein said duckbill valve is formed of elastomeric material.

4. A precision venting arrangement as recited in claim 3 wherein said distal end of said duckbill valve is disposed within said side passage of said housing to not extend into said housing lumen.

5. A precision venting arrangement as recited in claim 4 wherein said tubular housing has a “T” configuration with a transverse extending portion defining said side passage and a retaining cap received on said transverse portion to capture said venting valve.

6. A precision venting arrangement as recited in claim 5 and further comprising a pouch communicating with said second open end of said tubular housing for collecting urine from said medical tubing.
